(19) United States
(12) Patent Application Publication
(10) Pub. No.: US 2010/0063351 A1
(43) Pub. Date: Mar. 11, 2010

(54) SYSTEM FOR SURGICAL TREATMENT OF URINARY INCONTINENCE AND METHOD OF USE

(75) Inventors: Michael M. Witzmann, Minneapolis, MN (US); Emily C. Daley, Brooklyn Center, MN (US); Sarah J. Deitch, Stillwater, MN (US)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/553,100

(22) Filed: Sep. 3, 2009

(30) Foreign Application Priority Data
Sep. 4, 2008 (DK) PA 2008 01227

(51) Int. Cl. A61F 2/02

(52) U.S. Cl. 600/31

(57) ABSTRACT

A system configured to treat urinary incontinence includes an implant configured to support a urethra, a tensioning member connected to the implant, an elongated fixation member connected to the tensioning member, and an anchor connected to the elongated fixation member. The anchor includes a body extending between a penetration end and an introducer end, a cuff configured to slide relative to the body, and a plurality of expandable body segments coupled between the cuff and the penetration end. Movement of the cuff toward the penetration end radially expands the expandable body segments.

### FIELD OF THE INVENTION

The present invention relates generally to surgical devices and methods. The invention relates specifically to a system for surgical treatment of urinary incontinence and its method of use.

### BACKGROUND OF THE INVENTION

Urinary incontinence (UI) can range in severity from partial to complete loss of bladder control, and patients afflicted with UI can experience varying degrees of unintentional urine leakage. Incontinence conditions may change over time; and patients with light incontinence, for example, may experience minimal leakage during the occurrence of a provocative event, such as laughing or coughing, while those with heavy incontinence may experience continuous urine leakage.

Generally, UI is not considered a disease, but rather a symptom or side effect of another medical condition. Some causes of UI include prostate surgery and in particular total prostatectomies (in men), head and spinal cord injuries, infections, certain toxins such as excessive alcohol consumption, certain medications such as sedatives, and certain diseases such as cancer, Parkinson's disease, and multiple sclerosis. Indeed, incontinence can be caused simply by virtue of aging processes or even emotional distress.

As known to those in the urologic arts, there may be several types of UI and various treatments. Stress urinary incontinence, which is a common type of incontinence, may be characterized as urine leakage during a provocative event such as sneezing, laughing, lifting heavy objects, or when the patient engages in any type of exercise that puts pressure on the bladder. Urge urinary incontinence occurs when the patient wants to urinate but is incapable of exercising restraint until reaching a restroom. Additional types of incontinence may include overflow incontinence, which occurs when the quantity of urine exceeds the capacity of the patient's bladder, and functional incontinence, which occurs when the patient has knowledge of the need to urinate but simply cannot access a restroom quickly enough due to a physical obstruction or debilitation.

To treat UI, several options may be available. These treatments may include behavioral techniques, such as biofeedback, bladder training, and pelvic muscle exercises; and modifications of the patient's diet and fluid intake. With respect to the latter, it is known that eliminating or reducing intake of certain types of substances, such as caffeine and alcohol, may help to alleviate incontinence. There are, additionally, medications available such as dicyclomine (Bentyl), flavoxate (Urispas), hyoscyamine sulfate (Anaspaz), imipramine (Tofranil), oxybutynin

(Ditropan), tolterodine (Detrol), and propantheline (Pro-Banthine), phenylpropanolamine (Dexatrim), and pseudoephedrine (Sudafed), that may be helpful in controlling UI.

Surgery may additionally be an option to treat UI. Examples of commercially available surgical devices include suburethral slings such as the “InVance” device produced by American Medical Systems, Inc. (AMS) of Minneapolis, Minn. The “InVance” suburethral sling device is intended to provide structural support to the urethra for the treatment of stress urinary incontinence, thereby acting to retain urine within the bladder and not permit leakage through the urethra. Such “first generation” suburethral slings may not be easily used by urologists of ordinary skill in the art. For example, there may be problems with patients not being fully continent postoperatively due to the slings being anatomically fixated too loosely. Alternatively, there may also be problems with patients being put into retention postoperatively due to the devices being anatomically fixated too tightly.

Several companies have made attempts to progress the art of suburethral slings and their methods of use by providing so-called “minimally invasive” slings, which may utilize one or more mesh materials. One such company is Johnson & Johnson (J&J) which developed a sling known as “TVT-Secur” (TVT-S). It may be surgically implanted using only a single vaginal incision. The mesh sling is approximately 8 cm in length and has fixation tips fabricated from polyglactin 910 and polydioxanone suture yarn. The fixation tips of a TVT-S device may act to secure the mesh sling until tissue in-growth may occur along the length of the mesh—when the patient's anatomical tissues propagate within, through, and thus around pores of the mesh as is known with implantable mesh materials. To implant a TVT-S sling, a standard vaginal dissection and two small paraurethral dissections are performed. The sling has pre-attached introducers that may assist in placement to near a desired location within the two previously performed dissections. Near the desired location, the introducer may be detached from the sling and the other side of the sling may be placed. The sling may be placed in a “U” or “hammock” configuration. The incision may then be closed in standard fashion. Another “minimally invasive” suburethral sling is commercially available from the aforementioned AMS company. Its “Mini-Arc” suburethral sling device is approximately 8 cm in length with barb-like elements at each end. The barb-like elements may hold the sling in place until tissue in-growth occurs. Like the TVT-S device, a standard vaginal dissection and two small paraurethral dissections may be performed to place the sling. A trocar-like instrument may be attached to each barb-like element, to assist in placement near a correct tissue plane. After placement, the trocar may be released from the barb-like element; placement may then be made in the patient's contralateral side. The incision may then be closed in standard fashion.

As with the aforementioned “first generation” devices for treatment of UI, problems may also occur with use of “minimally invasive” slings. The J&J TVT-S devices, for example, may experience several problems. Its two aforementioned pre-attached introducers have rather bulky and sharp elements, which may cause excessive bleeding and hematomas—thereby making a procedure styled as “minimally invasive” in fact more invasive due to such complications. Also, fixation methods of TVT-S devices may be inadequate in that physicians may be unable to achieve continence in their patients due to faulty anchoring of the aforementioned fixation tips. The AMS “Mini-Arc” devices may function better than the J&J TVT-S devices; however, there may be two main drawbacks to these devices. First, the “Mini-Arc” device is relatively short which may impede a physician's ability to aim for a bony landmark as desired during implantation surgery. Desired repeatability of the surgical procedure from patient to patient may thereby diminished since anatomical landmarks may not be confidently or repeatably targeted, and instead the physician may need to rely upon experience and subjective judgment to place the barb-like elements near correct tissue planes. Second, anchoring and tensioning functions in the “Mini-Arc” device may be, disadvantageously, coupled. That is, in placing a second anchor, the physician might need to anticipate with nearly impossible foresight or luck that the desired anchor placement location will ultimately result in desired tensioning of the sling.

Aside from the aforementioned TVT-S and “Mini-Arc” devices, several attempts may have been made at providing “minimally invasive” UI sling-type systems. For example, U.S. Pat. No. 6,960,160 (Ser. No. 10/398,992) discloses an “apparatus and method for treating female urinary incontinence” that may require significant dissection and relatively invasive creation of a passage or tunnel in the patient's internal anatomical space to accommodate the device. U.S. Pat. No. 7,163,506 (Ser. No. 10/413,470) discloses a “device for the treatment of urinary incontinence” that may require a valve and conduit that are fluidly connected to an inflatable balloon for sling fixation. U.S. Pat. No. 7,229,404 (Ser. No. 10/524,861) discloses a “surgical prosthesis-forming device used to implant an organ support in a mammal” that may utilize a relatively long sling, coupled to a pulley-like device. Use of an invasive penetrating trocar or introducer may be required for implantation of the device. U.S. Pat. Applic. Pub. No. 2007/0162120 (Ser. No. 11/615,144) discloses a “surgical device forming a surgical prosthesis” that may use “bunched up” mesh—or a “ball of mesh”—as an anchoring means within tissue to which the device may be fixed, via tissue in-growth as aforedescribed. Consequently, this device may require use of a relatively large, hollow trocar device to surgically place the “bunched up” mesh anchor. PCT Pat. Applic. Pub. No. WO 2007/059199 (serial no. PCT/US06/044315) discloses a “sling anchor system” that may provide tensioning adjustment adjacent to the sling itself, relatively proximal to near the patient's anatomy to be supported rather than distally near the non-expandable anchor. PCT Pat. Applic. Pub. No. WO 2007/149555 (serial no. PCT/US07/014553) discloses “adjustable tension incontinence sling assemblies” that may rely upon anchors like those of the aforementioned “Mini-Arc” device and may require superficial exit points at a level of the patient's skin.

Regardless of construction, mode of operation, or invasiveness, various examples of suburethral sling devices, components, and methods of manufacture and use, may be described in (a) U.S. Pat. No. 6,626,916 (Ser. No. 09/445,011), U.S. Pat. No. 6,638,211 (Ser. No. 10/092,069), U.S. Pat. No. 6,960,160 as aforementioned (Ser. No. 10/398,992), U.S. Pat. No. 7,229,404 as aforementioned (Ser. No. 10/524,861), U.S. Pat. No. 7,285,086 (Ser. No. 11/190,295), and U.S. Pat. No. 7,297,102 (Ser. No. 11/190,601); (b) U.S. Pat. Applic. Pub. Nos. 2005/0043820 (Ser. No. 10/492,473), 2006/0030884 (Ser. No. 10/914,059), 2006/0041185 (Ser. No. 11/199,601), 2006/0058578 (Ser. No. 10/510,488), 2006/0205995 (Ser. No. 11/324,028), 2007/0162120 as aforementioned (Ser. No. 11/615,144), and 2007/0299300 (Ser. No. 11/854,049); and (c) PCT Pat. Applic. Pub. Nos. WO 2007/059199 as aforementioned (serial no. PCT/US06/044315), WO 2007/097994 (serial no. PCT/US07/004015), WO 2007/149348 (serial no. PCT/US07/014120), WO 2007/149555 as aforementioned (serial no. PCT/US07/014553), and WO 2007/149593 (serial no. PCT/US07/014780).

In general, several drawbacks may be experienced in utilizing suburethral slings per se to treat UI. For example, securing suburethral sling devices into position may require use of known anchoring devices such as, for example, bone screws, which could be difficult and time consuming to deploy, and which could result in significant patient discomfort—especially within a first few weeks following surgery. In this regard, it might be recognized among surgeons who perform implant procedures that sutures attached to bone anchors or bone screws, which are utilized to secure the devices into position, may break and that additional bone anchors or screws may be needed to secure the devices into position. In fact, sutures attached to bone anchors and bone screws may need to be individually re-tensioned several times before optimal device positioning and desired structural support to the urethra is achieved. In addition, surgical procedures and methods for implanting suburethral sling devices for treatment of UI utilizing known anchoring devices may lead to difficulties in positioning and fixation with desired anatomical tensioning.

The suburethral sling art might also be viewed as moving away from such invasive anchoring devices, to relatively less invasive fixation via tissue in-growth. The art might further be viewed as moving toward shortened sling lengths with, optionally, elimination of skin exit points above the patients' pubic anatomy—all in an effort to be less invasive or “minimally invasive”.

At present, so-called “minimally invasive” slings might have minimized sling lengths, but unfortunately they might have not led to lower trauma rates or increased out-patient use as desired. Such known “minimally invasive” slings may still require use of relatively large introducers and relatively large anatomical dissections. In some cases, placement of such slings may lead to even higher complication rates when compared to older, “first generation” slings.

Further, implantation of known suburethral sling devices—whether first generation or “minimally invasive”, may be complex and time consuming, and may produce unacceptable clinical outcomes.

Accordingly, there may be a need in the art for a system for surgical treatment of urinary incontinence of the present invention that might alleviate difficulties in placement, positioning, fixation, and anchoring, with desired anatomical tensioning when compared to known devices. Also, there may be a need in the art for a system for surgical treatment of urinary incontinence of the present invention that might be relatively less invasive when compared to known devices. Additionally, there may be a need in the art for a system for surgical treatment of urinary incontinence of the present invention that might be associated with lower trauma or complication rates while potentially increasing out-patient use. Further, there may be a need in the art for a system for surgical treatment of urinary incontinence of the present invention that might be implanted without complex or time consuming surgical procedures, and that might produce acceptable clinical outcomes.

### SUMMARY OF THE INVENTION

In accordance with basic aspects of the present invention, a system for surgical treatment of urinary incontinence could comprise an implantable material to support a urethra, having a first end portion, a central portion, and a second end portion. A first tensioning member could be connected to the first end portion of the implantable material, and a second tensioning member could be connected to the second end portion of the implantable material. A first elongated fixation member could be connected to the first tensioning member, and a second elongated fixation member could be connected to the second tensioning member. A first anchor could be connected to the first elongated fixation member and a second anchor could be connected to the second elongated fixation member.

Also in accordance with basic aspects of the present invention, a method of manufacturing an anchor for use in a system for surgical treatment of urinary incontinence could comprise steps of making a mold in a shape of an anchor, filling the mold with a selected semi-liquid material and curing it, and then removing the anchor thereby created from the mold.

Further in accordance with basic aspects of the present invention, a method of using a system for surgical treatment of urinary incontinence could comprise steps of providing (i) an implantable material to support a urethra, having a first end portion, a central portion, and a second end portion, (ii) a first tensioning member and a second tensioning member, (iii) a first elongated fixation member and a second elongated fixation member, (iv) a first anchor and a second anchor, and (v) an introducer. An anatomical dissection could be made in a patient to access internal anatomical space near both obturator foramen of the patient. The first anchor could be removably secured to the introducer, with (a) the first anchor connected to the first elongated fixation member, (b) the first elongated fixation member connected to the first tensioning member, and (c) the first tensioning member connected to the first end portion of said implantable material. The introducer, with the first anchor, the first elongated fixation member, the first tensioning member, and the first end portion of the implantable material, could be placed through the anatomical dissection in the patient, thereby entering the internal anatomical space near an obturator foramen of the patient with the first anchor and the first elongated fixation member. The introducer, with the first anchor, could be advanced into and through obturator tissue in the obturator foramen of the patient and deployed. The introducer could be removed from the first anchor, and withdrawn from the internal anatomical space and the anatomical dissection in the patient. The foregoing steps could be repeated with respect to placement of the second anchor, the second elongated fixation member, the second tensioning member, and the second end portion of the implantable material. The central portion of the implantable material could be manipulated for placement near a urethra of the patient. Optionally and individually, the first tensioning member connected to the first end of the implantable material, and the second tensioning member connected to the second end of the implantable material, could be manipulated to achieve desired tensioning of the implantable material near the urethra of the patient. The anatomical dissection in the patient could then be closed.

### BRIEF DESCRIPTION OF THE DRAWINGS

FIG. 1 is a schematic illustration of an example of a system for surgical treatment of urinary incontinence of the present invention, placed in a patient during implantation surgery.

FIG. 2 is an illustration of an example of tensioning components of the system depicted in FIG. 1.

FIG. 3 is an illustration of an alternative example of tensioning components of the system depicted in FIG. 1.

FIG. 4 is an illustration of an example of an anchor of the system shown in FIG. 1, depicting an un-deployed state.

FIG. 4a is an illustration of the example of an anchor shown in FIG. 4, depicting a deployed state.

FIG. 5 is an illustration of an alternative example of an anchor of the system shown in FIG. 1, depicting an un-inflated state.

FIG. 5a is an illustration of the example of an anchor shown in FIG. 5, depicting an inflated state.

FIG. 6 is an illustration of another alternative example of an anchor of the system shown in FIG. 1, depicting an un-deployed state.

FIG. 6a is an illustration of the example of an anchor shown in FIG. 6, depicting a deployed state.

### DETAILED DESCRIPTION OF THE INVENTION

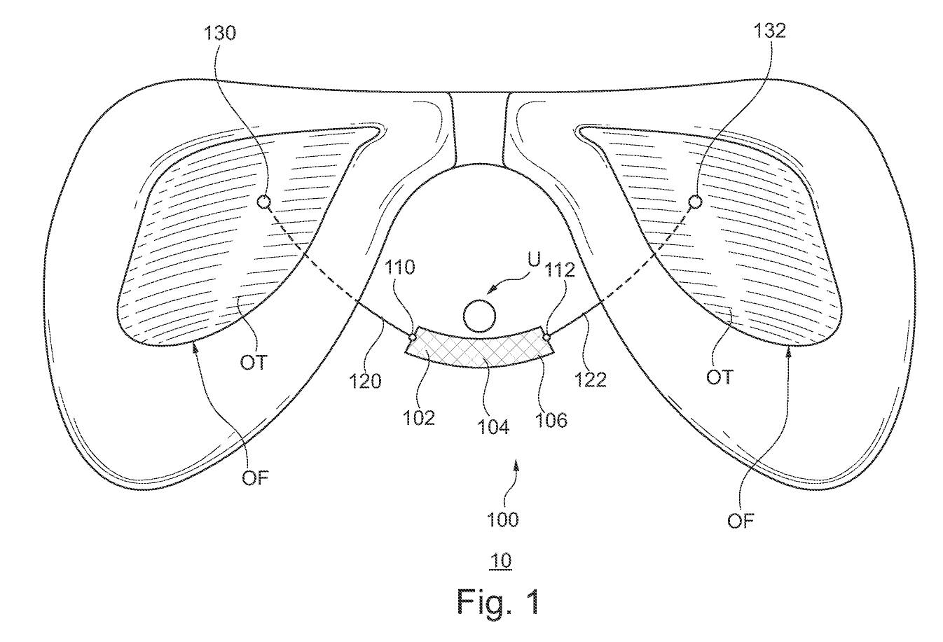

Illustrated in FIG. 1 is an example of a system for surgical treatment of urinary incontinence 10 (hereinafter, “system 10”) of the present invention. In this example, system 10 could include an implantable material 100 to support a urethra U, having a first end portion 102, a central portion 104, and a second end portion 106. System 10 could also include a first tensioning member 110 connected to first end portion 120 of implantable material 100, and a second tensioning member 112 connected to second end portion 106 of implantable material 100. System 10 could additionally include a first elongated fixation member 120 connected to first tensioning member 110, and a second elongated fixation member 122 connected to second tensioning member 112. System 10 could further include a first anchor 130 connected to first elongated fixation member 120 and a second anchor 132 connected to second elongated fixation member 122.

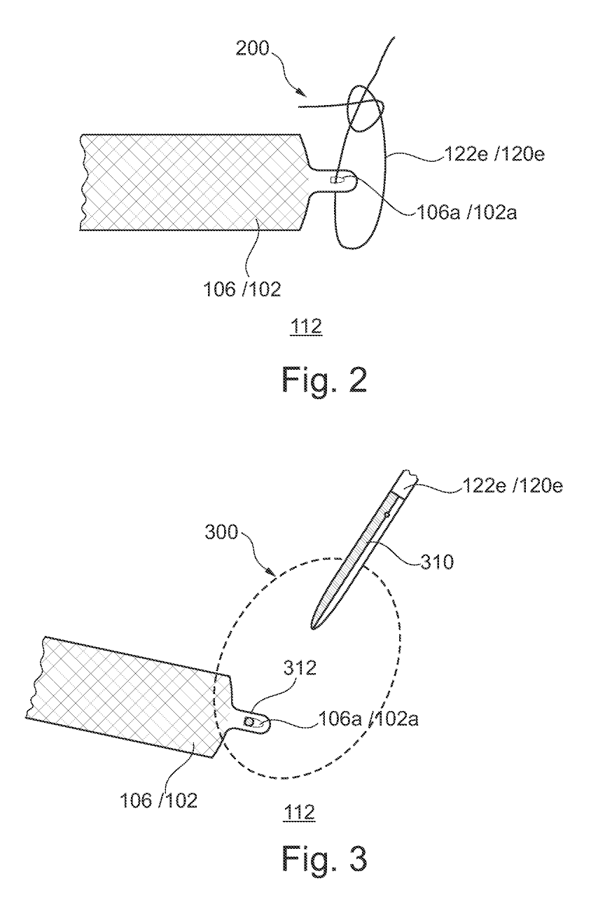

As illustrated in FIGS. 2 and 3, first and second tensioning members 110 and 112 could, individually or in combination, comprise a knot assembly 200 or a zip tie-like assembly 300. It is to be understood that although only tensioning member 112 is depicted in FIGS. 2 and 3, the example assemblies 200 and 300 as will be described could similarly be employed with opposite tensioning member 110. Thus, in FIG. 2, knot assembly 200 could include an end portion 122e or 120e of elongated fixation members 122 or 120 (not illustrated), respectively, that could be placed through an aperture 106a or 102a (not illustrated) in end portion 106 or 102 (not illustrated), respectively. Then, as will be described in use of system 10, end portion 122e or 120e could be tied into any desired knot when material 100 is approximately at a desired tension near urethra U, thereby tending to hold such tension in system 10 as desired. In FIG. 3, zip tie-like assembly 300 could include a relatively flat toothed or notched end portion 122e or 120e of elongated fixation members 122 or 120 (not illustrated), respectively. Toothed or notched end portion 122e or 120e (represented by teeth or notches 310 in the drawing) could be configured to be capable of being placed through an indexing aperture 106a or 102a in end portion 106 or 102 (not illustrated), respectively, that itself could have a complementary interior indexing surface 312 that is capable of incrementally advancing and securing teeth or notches 310 of end portion 122e or 120e, respectively, therewithin in “zip tie” fashion. Then, as will be described in use of system 10, end portion 122e or 120e could be placed within indexing aperture 106a or 102a, respectively, and pulled into successive engagement between notches 310 and complementary interior indexing surface 312 to approximately achieve a desired tension of material 100 near urethra U—thereby tending to hold such tension in system 10 as desired.

As may be desired for a particular system 10, elongated fixation members 120 and 122 could, individually or in combination, comprise a suitable medical grade mesh material or a medical grade suture material. Such a medical grade mesh material could be as described below, as an example of implantable material 100.

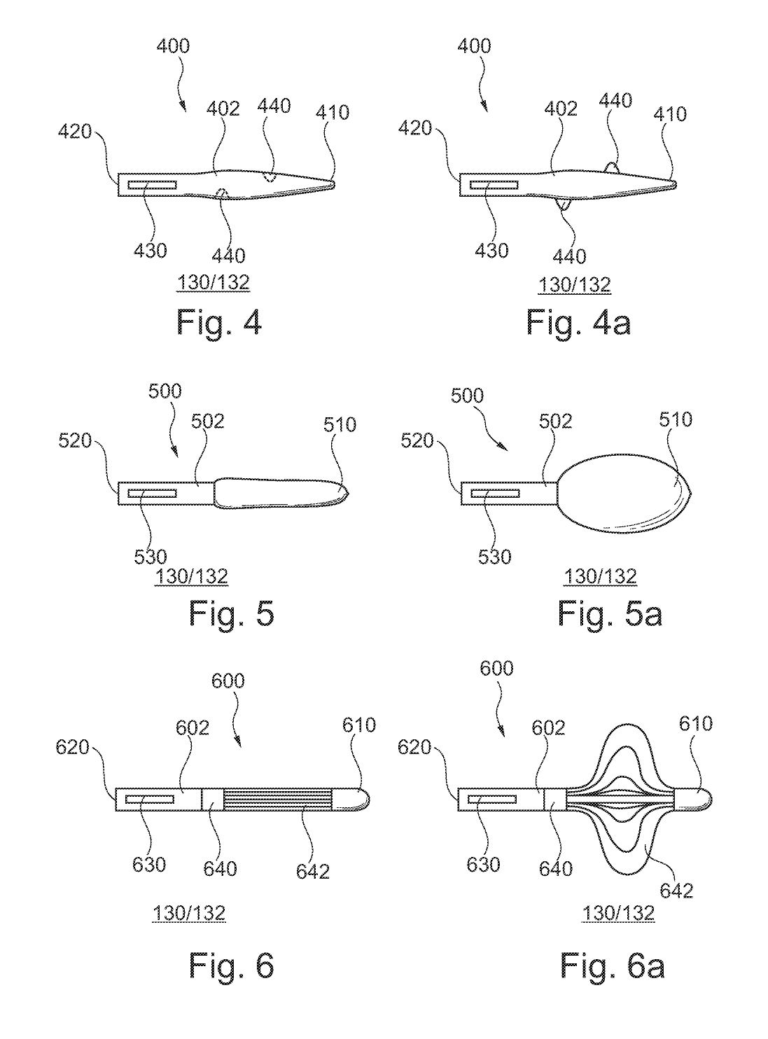

Turning now to FIGS. 4-6a, first and second anchors 130 and 132 could comprise, individually or in combination, for example, an expandable needle-like element (FIGS. 4 and 4a), an expandable balloon-like element (FIGS. 5 and 5a), or an expandable segmented element (FIGS. 6 and 6a).

In the example shown in FIGS. 4-4a, anchor 130/132 could be an expandable needle-like element 400 that could have a generally cylindrical or conical body 402. Body 402 could be further defined, lengthwise, by a relatively pointed tissue penetrating end 410 and an introducer receiving end 420. Body 402 could include a channel (not illustrated) in introducer receiving end 420 for removably securing a tip of an introducer (also not illustrated) therewithin as will be described relative to use of system 10. Body 402 could also include a slot 430 for placement and securing of portions of elongated fixation members 120 or 122 therethrough, as will also be described relative to use of system 10. Body 402 could further include one or more apertures to accommodate one or more deployable barbs 440 for immobilizing and fixing expandable needle-like element 400 in anatomical tissue, as will also be described relative to use of system 10. Any suitable means (not illustrated) for deploying barbs 440 may be utilized upon placement of element 400 in a desired anchoring location, such as by, for example, mechanical or electro-mechanical actuation. Dimensions of expandable needle-like element 400 could be, for example, 0.300 inches (0.76200 cm) in length by 0.062 inches (0.15748 cm) in diameter, while the channel in introducer receiving end 420 could be 0.045 inches (0.1143 cm) to 0.050 inches (0.1270 cm) in diameter; and slot 430 could be 0.110 inches (0.2794 cm) in length by 0.020 inches (0.0508 cm) in width. Element 400 could be constructed using any suitable techniques as will be described below.

In the example shown in FIGS. 5-5a, anchor 130/132 could be an expandable balloon-like element 500 that could have a generally cylindrical body 502 and tissue penetrating end portion 510 in an un-inflated state as shown in FIG. 5. Body 502 could be further defined, lengthwise, by an introducer receiving end 520. Body 502 could include a channel (not illustrated) in introducer receiving end 520 for removably securing a tip of an introducer (also not illustrated) therewithin as will be described relative to use of system 10. As will also be described relative to use of system 10, body 502 could also include a slot 530 for placement and securing of portions of elongated fixation members 120 or 122 therethrough. After penetrating anatomical tissue in which anchor 130/132 is to be fixed, expandable balloon-like element 500 could be inflated as shown in FIG. 5a such that anchor 130/132 could be immobilized and fixed in the anatomical tissue thereby. Any suitable means (not illustrated) for inflating expandable balloon-like element 500 could be utilized upon placement of element 500 in a desired tissue anchoring location, such as by, for example, pneumatic, electro-pneumatic, hydraulic, or electro-hydraulic actuation. Dimensions of expandable balloon-like element 500 could be, for example, 0.300 inches (0.7620 cm) in length by 0.085 inches (0.2159 cm) in diameter, while the channel in introducer receiving end 520 could be 0.045 inches (0.1143 cm) to 0.050 inches (0.1270 cm) in diameter; and slot 530 could be 0.110 inches (0.2794 cm) in length by 0.020 inches (0.0508 cm) in width. Element 500 could be constructed using any suitable techniques as will be described below.

Lastly, in the example shown in FIGS. 6-6a, anchor 130/132 could be an expandable segmented element 600 that could have a generally cylindrical body 602. Body 602 could be further defined, lengthwise, by a relatively pointed tissue penetrating end 610 and an introducer receiving end 620. Body 602 could include a channel (not illustrated) in introducer receiving end 620 for removably securing a tip of an introducer (also not illustrated) therewithin as will be described relative to use of system 10. Body 602 could also include a slot 630 for placement and securing of portions of elongated fixation members 120 or 122 therethrough, as will also be described relative to use of system 10. Body 602 could further include a slidable cuff 640 and one or more expandable body segments 642 connected proximally to cuff 640 and distally to tissue penetrating end 610, for immobilizing and fixing expandable segmented element 600 in anatomical tissue as will also be described relative to use of system 10. Element 600 could remain in a generally cylindrical configuration until desired deployment, whereupon movement of cuff 640 toward end 610 could cause one or more expandable body segments 642 to bend or otherwise expand outwardly in explosive-like fashion—thereby effectively forming a disc-type anchor while in a deployed state. Any suitable means (not illustrated) for deploying segments 642 may be utilized upon placement of element 600 in a desired anchoring location, such as by, for example, a physician's manipulation of cuff 640 or even mechanical or electro-mechanical actuation of cuff 640. Dimensions of expandable segmented element 600 could be, for example, 0.300 inches (0.76200 cm) in length by 0.085 inches (0.2159 cm) in diameter, while the channel in introducer receiving end 620 could be 0.045 inches (0.1143 cm) to 0.050 inches (0.1270 cm) in diameter; and slot 630 could be 0.110 inches (0.2794 cm) in length by 0.020 inches (0.0508 cm) in width. Element 600 could be constructed using any suitable techniques as will be described below.

It is to be understood that, as shown particularly in the example of FIG. 1, a system for surgical treatment of urinary incontinence of the present invention could advantageously employ a relatively short length of implantable material 100, relatively thin elongated fixation members 120 and 122, and relatively remote placement of anchors 130 and 132, as may be appreciated with respect to use of the system of the present invention as described below.

Although not illustrated, it is to be understood that certain components of a system for surgical treatment of urinary incontinence of the present invention, such as anchors 130 or 132, could be manufactured using any suitable materials (e.g., semi-liquid materials and metals such as stainless steel and Nitinol) and fabrication techniques. For example, a mold could be made in a shape of anchor 130 as embodied in expandable balloon-like element 500, most notably including body 502, tissue penetrating end portion 510, introducer receiving end 520 including the aforementioned channel, and slot 530. The mold could be filled with a selected semi-liquid material. The semi-liquid material could then be cured or cooled (hereinafter, individually or collectively, referred to as “cured” or “curing”) and expandable balloon-like element 500 thereby created could then be removed from the mold. Alternatively, a mold could be made in a substantially solid shape of, for example, body 400 of expandable needle-like element 400. Then, the channel in introducer receiving end 420, slot 430, and the apertures for deployable barbs 440 could be formed by any suitable technique such as by machining, drilling, or milling processes; and provision of barbs 440 could be made by constructing a spring-loaded or electro-mechanical sub-assembly incorporated into body 400. The semi-liquid material could be, as desired or suitable for a particular manufacturing process, any suitable medical grade thermoplastic/thermoset and/or polymer-like material, of desired durometer for desired hardness, such as, for example, polyolefin, polypropylene, polyethylene, polyurethane, polycarbonate, polysulfone, so-called “ABS” polymer material, nylon material, or silicone; or combinations of these materials. Curing (or “cycle”) times and temperatures, along with a particular selection of the material or materials, could determine optimal characteristics of thickness, hardness, and durability of anchors 130 or 132. It is to be understood that a particular embodiment of anchors 130 or 132, such as the examples of expandable needle-like element 400, expandable balloon-like element 500, and expandable segmented element 600, could be produced using any suitable materials and fabrication techniques as aforementioned, depending upon particular requirements and configurations of each. As such, suitable materials and fabrication techniques could employ several different materials (e.g., polyethylene, nylon, and silicone) and several different fabrication steps (e.g., molding, machining, and construction of a spring-loaded sub-assembly) for production of an anchor 130 or 132. It is to be appreciated that, although again not illustrated herein, anchors 130 or 132 could also be manufactured using any suitable fabrication techniques such as transfer molding or injection molding.

With reference now to all of the drawings, an example of a method of using a system for surgical treatment of urinary incontinence of the present invention, in a female patient, may include the following steps.

First and second anchors 130 and 132 (e.g., 400, 500, or 600) could be provided to a physician, along with a suitable implantable material 100 and a suitable introducer. Implantable material 100 could have a first end portion 102, a central portion 104, and a second end portion 106. Material 100 could have a length of about 4 cm and a width of about 11 mm. Suitable implantable material and a suitable introducer could be, for example, ARIS® brand suburethral sling material and an ARIS® brand curved introducer that are both commercially available from Coloplast A/S. The physician could then make vaginal dissection (or “midline dissection”; not illustrated) in the female patient to access internal anatomical space near the patient's obturator foramen OF (as shown in FIG. 1).

Referring to FIG. 2 and, alternatively, FIG. 3, a selected portion of first elongated fixation member 120 could then be placed through slot (430, 530, or 630) of first anchor 130 (400, 500, or 600) such that elongated fixation member 120 is secured within slot (430, 530, or 630). An opposite portion of first elongated fixation member 120 could then be connected to first tensioning member 110 (e.g., 200 or 300, as illustrated in FIGS. 2 and 3 relative to similar, opposing second tensioning member 112 and second elongated fixation member 122) thereby connecting first tensioning member 110 to first end portion 102 of implantable material 100. Although not shown in the drawings, the introducer could be removably coupled to anchor 130 by way of pushing a tip of the introducer into the channel of anchor 130 at introducer receiving end (420, 520, or 620). The introducer—with anchor 130 coupled to it and sequentially connected first elongated fixation member 120 and first end portion 102 of material 100 via first tensioning member 110 as aforedescribed—could then be placed through the vaginal dissection so that the introducer, anchor 130, and material 100 could all enter the internal anatomical space near the patient's obturator foramen OF. The introducer, with anchor 130 and a portion of first elongated fixation member 120 connected to anchor 130 at slot (430, 530, or 630), could then be advanced into and through obturator tissue OT in the obturator foramen OF adjacent to a posterior side of an ischiopubic ramus. Commonly, an audible and/or tactile “pop” is noticed when obturator tissue is so penetrated. The physician could then cause anchor 130 to deploy (e.g., FIG. 4a), inflate (e.g., FIG. 5a), or expand outwardly (e.g., FIG. 6a)—collectively, “deploy”—thus tending to inhibit movement of anchor 130 back through penetrated obturator tissue OT and thereby effectively anchoring a portion of first elongated fixation member 120 adjacent to anchor 130 in obturator foramen OF. The introducer could then be removed by pulling its tip outwardly from the channel in introducer receiving end (420, 520, or 620) of anchor 130; and subsequently the introducer could be withdrawn from the internal anatomical space and the vaginal dissection in the patient. The aforedescribed steps could then be repeated on the patient's contralateral side with respect to placement of second anchor 132 as illustrated in FIG. 1. Central portion 104 of material 100 could then be manipulated by the physician through the vaginal dissection to ensure placement of central portion 104 near a urethra U of the patient so that urethra U may be supported or even compressed as may be necessary for a particular UI treatment. Optionally and individually, the physician could also manipulate first and second tensioning members 110 and 112 (e.g., assemblies 200 or 300) to achieve desired tensioning of implantable material 100 near urethra U. Finally, the vaginal dissection could be closed by any suitable surgical technique such as suturing, thereby concluding the implantation surgery.

It is to be appreciated that although this example of a surgical method utilizing system 10 has been described relative to female patients, it could of course also be performed in male patients with corresponding and/or alternative surgical steps (e.g., a perineal incision in place of a vaginal dissection).

It is to be also appreciated that the aforedescribed example of a method utilizing the system for surgical treatment of urinary incontinence of the present invention is intended to be a “minimally invasive” treatment. In this method it is to be understood, with particular reference to FIG. 1, that material 100 could “cradle” the patient's urethra U. Elongated fixation member 120 could be effectively fixed in one obturator foramen OF, via one anchor 130 that penetrates obturator tissue OT and traverses the patient's internal anatomical space to first tensioning member 110. Central portion 104 of material 100 could then be manipulated to be approximately under urethra U. On a contralateral side, elongated fixation member 122 could then be effectively fixed in the obturator foramen OF via the other anchor 132 that penetrates obturator tissue OT and traverses the patient's internal anatomical space to second tensioning member 112. Central portion 104 of material 100 could then again be manipulated to be approximately under urethra U. Thus, material 100 could effectively reside between urethra U and the patient's anterior vaginal wall adjacent to the vaginal dissection performed by the physician. This “cradle” of material 100 could serve to support urethra U in response to, for example, a stress event such as coughing or laughing, thereby tending to prevent, for example, stress UI.

Material 100 could have an ability, with respect to use of zip tie-like assembly 300, for example, to be tensioned as aforementioned by carefully and incrementally advancing toothed or notched end portion 122e and/or 120e in corresponding indexing aperture 106a and/or 102a. With reference to a side of system 10 corresponding to anchor 132, as notched end portion 122e is so advanced within indexing aperture 106a, a length of elongated fixation member 122 between anchor 132 and tensioning member 112 decreases, which thereby could provide desired increased tension against, or increased lifting force under, urethra U. Of course, this tensioning process could be performed on each side of system 10 corresponding to anchors 132 and 130, either alone, consecutively, or concurrently with respect to each other. It is to be understood that material 100 could also have an ability to be similarly tensioned with respect to use of example knot assembly 200, as aforementioned. Further, it is to be understood that knot assembly 200 could be employed on one side of a particular system 10 and that zip tie-like assembly 300 could be employed on a contralateral side of that same system 10, depending upon a physician's preference.

Also, it has been discovered that upon occurrence of so-called “tissue in-growth” of material 100 after the surgery is completed and during the patient's healing process, anchors 130 and 132 might then be not needed to fix material 100 in the patient; and therefore anchors 130 and 132 could be made of a suitable medical grade bioresorbable material.

It is to be additionally appreciated that the foregoing example of a method to treat UI provides an anchor placement step that, notably unlike the AMS “Mini-Arc” device, is intentionally “decoupled” from tensioning of the suburethral sling material. In the foregoing example, both anchors could be placed and checked for desired positioning. Thereafter, when the anchors are in desired positions, tensioning of the sling material could then be performed. In the “Mini-Arc” device, however, tensioning is in fact “coupled” to anchor fixation—distal or proximal placement of a second anchor with respect to the urethra dictates the tension of the sling material.

It is to be further appreciated and understood that the foregoing example of a method to treat UI could provide an ability for the physician to place the anchors near discrete “bony landmarks”. This ability could be another distinguishing factor from the AMS “Mini-Arc” device, which intentionally utilizes a relatively short length of sling material and thereby impedes a physician from placing anchors near such “bony landmarks”. Rather, a physician implanting an AMS device must find a suitable location for anchor placement by subjective reliance on past surgical experience. Also, unlike the TVT-S device which could embody a relatively large configuration, it is also to be appreciated and understood that the system of the present invention could have an ability to minimize dissection and trauma to pelvic tissues by employing expandable tissue anchors that could be attached via small diameter and variable length fixation members to a relatively small length of implantable material that could support or compress the urethra. The attachment of the fixation members to the implantable material could provide length adjustment for tensioning via the tensioning members as aforesaid. The anchors could be placed by a relatively small diameter introducer that minimally penetrates through anatomical tissue to a desired location for fixation of each anchor. Once at the desired location, the anchors could be deployed such that expansion thereof could immobilize them. Since the anchors could be expandable, damage to tissue could thus be minimized by use of such small anchors and introducer that are not expanded until in the correct location. Additionally, the fixation members that attach to the anchors could be of even smaller diameter than the introducer, thereby causing no additional tissue trauma during implantation surgery. A length of the implantable material could then be reduced to support just a space near the urethra. As such, the dissection could be performed to be only large enough to accommodate the reduced material length that actually cradles the urethra. The relative dissection size could thus be decreased since relatively long fixation arms or relatively large anchor barbs—as utilized in other devices—do not require accommodation in the system of the present invention.

Although not illustrated in the drawings, it is to be appreciated that an anchor provided in accordance with the present invention could have a hole or aperture in its penetrating end so that a tip of an introducer could protrude therefrom (e.g., through a channel that could be provided completely through the anchor's body).

It is to be appreciated from the foregoing disclosure that the present invention uniquely and advantageously satisfies the long-felt need for a system for surgical treatment of urinary incontinence that may be easier to surgically implant and secure into position than known devices, and that may be simply constructed, manufactured at relatively low cost, and easy to use when compared to known devices.

It will be appreciated by those in the surgical arts that novel aspects of the tensioning members and anchors of the present invention could be capable of use in, and beneficial to, virtually any anatomical tensioning and anchoring per se—even outside of UI treatment technologies.

While the present invention has been particularly shown and described with reference to the accompanying specification and drawings, it will be understood however that other modifications thereto are of course possible; and all of which are intended to be within the true spirit and scope of the present invention. It should be appreciated that (i) components, dimensions, shapes, and other particulars of example embodiments of the invention aforedescribed may be substituted for others that are suitable for achieving desired results, (ii) various additions or deletions may be made thereto, and (iii) features of the foregoing examples may also be made in combinations thereof. It is also to be understood in general that any suitable alternatives may be employed to provide the system for surgical treatment of urinary incontinence of the present invention, its manufacturing method, and its method of use.

Lastly, of course, the choice of compositions, sizes, and strengths of various aforementioned elements of the present invention are all a matter of design choice depending upon intended uses thereof.

Accordingly, these and other various changes or modifications in form and detail of the present invention may also be made therein, again without departing from the true spirit and scope of the invention as defined by the appended claims.

What is claimed:

1. A system configured to treat urinary incontinence, the system comprising:
an implant configured to support a urethra;
a tensioning member connected to the implant;
an elongated fixation member connected to the tensioning member; and
an anchor connected to the elongated fixation member, the anchor comprising a body extending between a penetration end and an introducer end, a cuff configured to slide relative to the body, and a plurality of expandable body segments coupled between the cuff and the penetration end;
wherein movement of the cuff toward the penetration end radially expands the expandable body segments.

2. The system of claim 1, wherein the tensioning member comprises one of a knot assembly and a zip tie assembly.

3. The system of claim 2, wherein the tensioning member is configured to adjust a length of the elongated fixation member and thus adjust a tension of the implant relative to the urethra.

4. The system of claim 1, wherein the elongated fixation member comprises one of a mesh material and a suture material.

5. The system of claim 1, wherein the cuff comprises a ring disposed around the body of the anchor.

6. The system of claim 1, wherein the cuff is configured to be moved manually.

7. The system of claim 1, wherein the cuff is configured to be moved electro-mechanically.

8. The system of claim 1, wherein the anchor comprises a substantially cylindrical shape when the cuff is located adjacent to the penetration end and a substantially disc shape when the cuff is moved distally toward the penetration end.

9. The system of claim 1, comprising:
a first tensioning member connected to a first end of the implant and a second tensioning member connected to a second end of the implant;
a first elongated fixation member connected to the first tensioning member and a second elongated fixation member connected to the second tensioning member; and
a first anchor connected to the first elongated fixation member and a second anchor connected to the second elongated fixation member.

10. The system of claim 9, wherein each tensioning member is configured to adjust a length of the elongated fixation member to which it is connected and thus adjust a tension of the implant relative to the urethra.

11. A method of surgically treating urinary incontinence in a patient, the method comprising:
inserting an anchor into a membrane of an obturator foramen of the patient;
moving a component of the anchor in an axial direction and deploying a plurality of expandable body segments in a radial direction, thus securing the anchor to the membrane;
placing a support member relative to a urethra of the patient; and
connecting the support member to the anchor.

12. The method of claim 11, comprising sliding a cuff of the anchor toward a penetration end of the anchor.

13. The method of claim 11, wherein the cuff is disposed around a body of the anchor and is slideable relative to the body of the anchor.

14. The method of claim 11, further comprising:
connecting a tensioning member between the support member and the anchor; and
adjusting tension of the support member relative to the urethra with the tensioning member.

15. A method of surgically treating urinary incontinence in a patient, the method comprising:
inserting an anchor into a membrane of an obturator foramen of the patient;
moving a component of the anchor in an axial direction and deploying a plurality of expandable body segments in a radial direction, thus securing the anchor to the membrane;
placing a support member relative to a urethra of the patient;
connecting a tensioning member between the support member and the anchor; and
adjusting tension of the support member relative to the urethra with the tensioning member.

16. The method of claim 15, comprising inserting an anchor into a membrane of each of a pair of obturator foramen of the patient; and
connecting a first tensioning member between the support member and a first one of the anchors and connecting a second tensioning member between the support member and a second one of the anchors; and
adjusting tension of the support member relative to the urethra with the first and second tensioning members.

17. The method of claim 16, comprising adjusting tension of the support member relative to the urethra by selectively shortening each of the first and second tensioning members.
